(19) United States
(12) Patent Application Publication
(10) Pub. No.: US 2022/0379095 A1
(43) Pub. Date: Dec. 1, 2022

(54) NEEDLE ASSEMBLY FOR VISUALIZATION OF FLUID MOVEMENT

(71) Applicant: Becton, Dickinson and Company, Franklin Lakes, NJ (US)

(72) Inventors: Blake Harper, Bountiful, UT (US); Jordan Diamond, Salt Lake City, UT (US); Huy Tran, Riverton, UT (US)

(21) Appl. No.: 17/731,119

(22) Filed: Apr. 27, 2022

Related U.S. Application Data
(60) Provisional application No. 63/194,054, filed on May 27, 2021.

(51) Int. Cl. A61M 25/06; A61M 25/09

(52) U.S. Cl. CPC A61M 25/0693; A61M 25/09041; A61M 25/0606

(57) ABSTRACT

A needle assembly of a catheter system may include one or more features to facilitate flashback confirmation of catheterization while also preventing buildup of positive pressure within a flashback chamber of the needle assembly. The needle assembly may include a plug, which may include an aperture for receiving an introducer needle. A proximal opening of the introducer needle may be accessible from the proximal end of the needle hub. The plug may include a vent between an outer surface of the plug and an inner surface of the needle hub to prevent the buildup of positive pressure. The plug may include an extended distal end that reduces a volume of the flashback chamber. The plug may include a window in fluid communication with a notch of the introducer needle. Blood may flow from the notch into the flashback chamber with a reduced volume via the window of the plug.

### RELATED APPLICATIONS

This application claims the benefit of U.S. Provisional Patent Application No. 63/194,054, filed on May 27, 2021, entitled NEEDLE ASSEMBLY FOR VISUALIZATION OF FLUID MOVEMENT, which is incorporated herein in its entirety.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is a peripheral intravenous catheter (“PIVC”) that is over-the-needle. As its name implies, the PIVC that is over-the-needle may be mounted over an introducer needle having a sharp distal tip. The PIVC and the introducer needle may be assembled so that the distal tip of the introducer needle extends beyond a distal tip of the PIVC with the bevel of the needle facing up away from skin of the patient. The PIVC and the introducer needle are generally inserted at a shallow angle through the skin into vasculature of the patient. The PIVC may further be used with a guidewire for precise placement of the distal tip of the PIVC at a desired location with the patient vasculature. After successful placement of the distal tip of the PIVC within the vasculature, the guidewire may be withdrawn in conjunction with the introducer needle.

In order to verify proper placement of the introducer needle and/or the distal tip of the PIVC in the vasculature, a user generally confirms that there is flashback of blood within a catheter system that includes the PIVC. The user visualizes the flashback of blood within the catheter system to determine the introducer needle is within the vasculature. Flashback visualization in a guidewire assisted PIVC is compromised by reduced flow rate of blood entering the PIVC due to placement of the guidewire in a fluid pathway of the catheter. Flashback visualization in a guidewire assisted PIVC may be achieved via a flashback chamber, however user perception of fluid advancement within the flashback chamber is difficult due to the large volume of the flashback chamber and the slow flow rate of blood entering the flashback chamber. Further, if the catheter system includes the guidewire assisted PIVC system and is closed, presence of the guidewire in the PIVC and the closed nature of the catheter system may create positive pressures within the PIVC and the flashback chamber, thereby preventing the blood from entering the flashback chamber.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access systems and related devices and methods. In some embodiments, a catheter system may include a catheter assembly and/or a needle assembly. In some embodiments, the catheter assembly may include a catheter adapter and a catheter extending distally from a distal end of the catheter adapter. In some embodiments, the catheter may include a peripheral intravenous catheter (PIVC), a midline catheter, or a peripherally inserted central catheter. In some embodiments, a proximal end of the catheter adapter may be configured to receive or otherwise couple to a needle assembly.

In some embodiments, the needle assembly may include various features to facilitate use of a guidewire. In some embodiments, the needle assembly may include various features to facilitate flashback confirmation of proper intravenous catheterization. In some embodiments, the needle assembly may include various features to prevent buildup of positive pressure within a flashback chamber of the needle assembly.

In some embodiments, the needle assembly may include a needle hub, which may include a proximal end, a distal end, and an interior interposed therebetween. In some embodiments, the proximal end of the needle hub may include a proximal opening. In some embodiments, the distal end of the needle hub may include a distal opening. In some embodiments, the needle assembly may include a plug and an introducer needle.

In some embodiments, the plug may include an outer surface, which may include a first portion in contact with an inner surface of the interior. In some embodiments, the plug may include a distal end, a proximal end, and an aperture interconnecting the distal end of the plug and the proximal end of the plug. In some embodiments, the first portion may include a vent extending between the distal end of the plug and the proximal end of the plug and providing a fluid pathway between the outer surface of the plug and the inner surface of the interior. In some embodiments, the fluid pathway may interconnect the interior and the proximal opening of the needle hub.

In some embodiments, the introducer needle may include a distal end and a proximal end. In some embodiments, the proximal end of the introducer needle may be secured to the proximal end of the needle hub via the aperture, and the distal end of the introducer needle may extend beyond the distal end of the needle hub. In some embodiments, a proximal opening of the introducer needle may be accessible at the proximal end of the needle hub such that a guidewire may be threaded through a lumen of the introducer needle via the proximal opening of the introducer needle and extend out through a distal opening of the introducer needle. In some embodiments, the introducer needle may include a notch forming a pathway through a sidewall of the introducer needle and in fluid communication with the lumen. In some embodiments, the flashback chamber may be in fluid communication with the notch and may define a space between the inner surface and a second portion of the outer surface.

In some embodiments, the flashback chamber of the needle hub may be positioned between the distal end of the plug and the distal end of the needle hub. In some embodiments, the fluid pathway may include a cross-sectional area that permits the passage of air but not of blood. In some embodiments, the cross-sectional area may be between 0.0001 and 0.0003 inches. In some embodiments, the needle hub may include a fluid tight seal between the distal end of the plug and the inner surface of the interior.

In some embodiments, the distal end of the plug may include a window, which may be in fluid communication with the flashback chamber. In some embodiments, the notch of the introducer needle may be positioned within the window such that the notch is in fluid communication with the flashback chamber via the window. In some embodiments, a fluid tight seal may be provided between an outer surface of the introducer needle and the aperture of the plug such that blood is prevented from flowing therebetween.

In some embodiments, the second portion of the outer surface of the plug may include an outer diameter that is less than an inner diameter of the interior. In some embodiments, the notch of the introducer needle may be positioned within the flashback chamber when the proximal end of the introducer needle is aligned with the proximal end of the plug or the proximal end of the needle hub. In some embodiments, a distal end of the plug may be chamfered inwardly from the proximal end of the plug to form a chamfered surface. In some embodiments, the chamfered surface may assist in aligning and guiding the introducer needle through the plug during assembly. Various other proximal ends and/or distal ends of the components of the needle assembly may be similarly chamfered to assist alignment during assembly.

In some embodiments, a gap is provided between the proximal end of the plug and the inner surface of the interior in proximity to the proximal opening of the needle hub. In some embodiments, the gap may permit passage of air after the air passes through the vent of the plug. In some embodiments, the gap may be interposed between the vent and the proximal opening of the needle hub.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is an upper perspective view of an example catheter system, illustrating an example needle assembly, according to some embodiments;

FIG. 2 is a cross-sectional view of the needle assembly, according to some embodiments;

FIG. 3 is an exploded cross-sectional view of the needle assembly, according to some embodiments;

FIG. 4 is an upper perspective view of an example plug, according to some embodiments;

FIG. 5 is a cross-sectional view of a portion of the needle assembly, according to some embodiments;

FIG. 6 is a cross-sectional view of another example needle assembly, according to some embodiments;

FIG. 7 is a partial cutaway view of the other needle assembly, according to some embodiments; and

FIG. 8 is a cross-sectional view of the other needle assembly, according to some embodiments.

### DESCRIPTION OF EMBODIMENTS

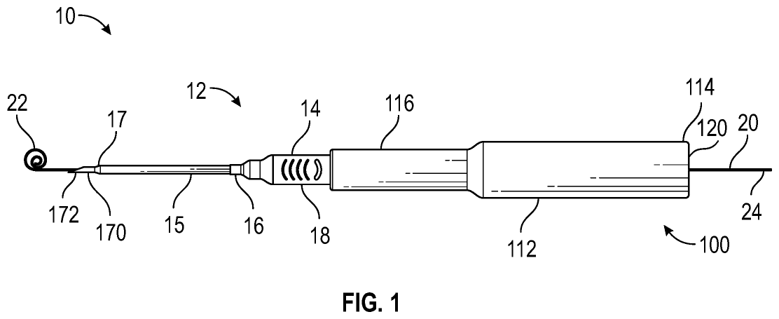

As used in the present disclosure, the term “proximal” refers to the portion of a structure closer to a user, and the term “distal” refers to the portion further from the user. Referring now to FIG. 1, a catheter system 10 is illustrated, according to some embodiments. In some embodiments, the catheter system 10 may be guidewire assisted or in other words, configured for use with a guidewire. In some embodiments, the catheter system 10 may include a catheter assembly 12, which may include a catheter adapter 14.

In some embodiments, the catheter adapter 14 may include a distal end 16. In some embodiments, the catheter assembly 12 may include a catheter 15, which may extend distally from distal end 16. In some embodiments, the catheter 15 may include a PIVC.

In some embodiments, the catheter adapter 14 may include a proximal end 18, which may be configured to selectively connect to a needle assembly 100 of the catheter system 10. In some embodiments, following catheterization of a patient, the catheter adapter 14 may be separated from needle assembly 100, and needle assembly 100 may be disposed.

In some embodiments, the needle assembly 100 may include a needle hub 112, which may include a distal end 116 configured to selectively receive and retain the proximal end 18 of the catheter adapter 14 during catheterization. In some embodiments, the catheter system 10 may further include additional known intravenous catheter assembly components and features, such as, for example, a needle shield, a securement platform, and various gripping surfaces (not illustrated).

In some embodiments, the needle assembly 100 may include a needle 170, which may include an introducer needle. In some embodiments, the needle 170 may include a proximal end secured to the needle hub 112, and a sharpened distal end 172 that extends distally beyond the distal tip 17 of the catheter 15 when the needle assembly 100 is secured to the catheter adapter 14.

In some embodiments, the catheter system 10 may include or may be compatible for use with a guidewire 20. In some embodiments, the guidewire 20 may be inserted through a lumen of the needle 170 such that a distal end 22 of the guidewire 20 may extend beyond the sharpened distal end 172 and such that a proximal end 24 of guidewire 20 extends proximally from a proximal opening 120 in a proximal end 114 of needle hub 112. As such, a user may manipulate the distal end 22 of guidewire 20 by directly contacting and controlling the proximal end 24 of the guidewire 20.

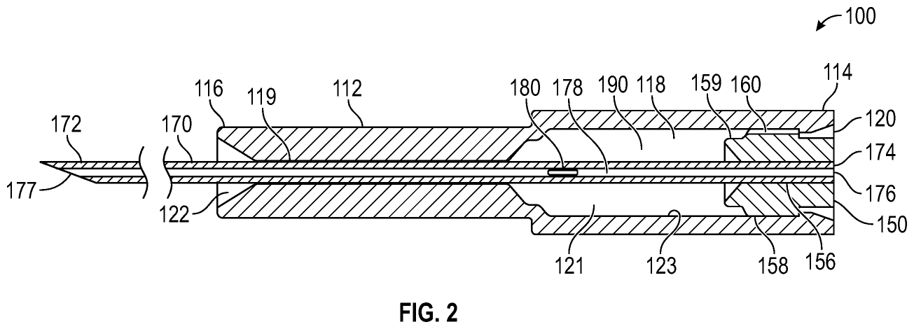

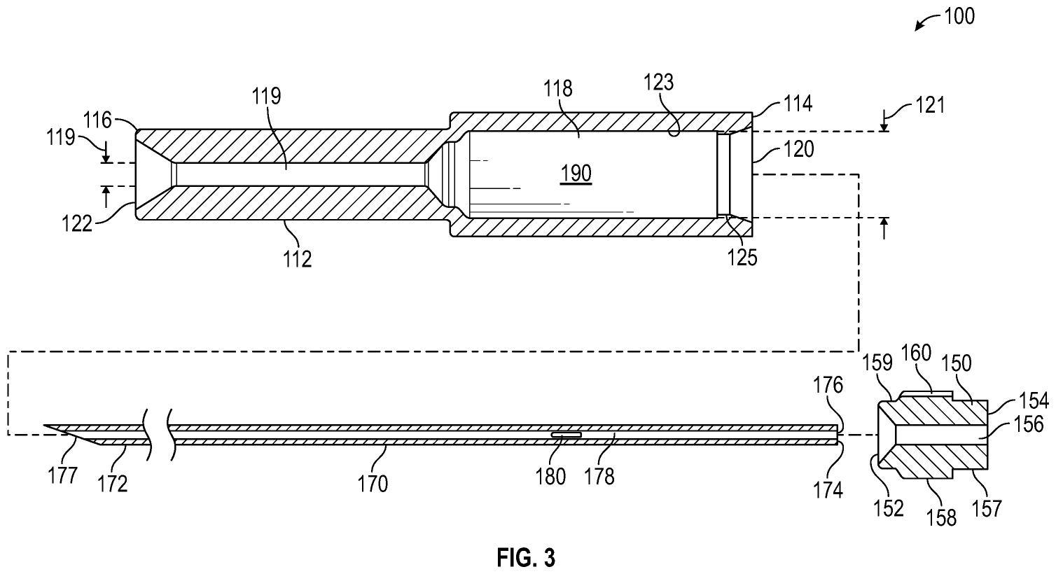

Referring now to FIGS. 2-5, the needle assembly 100 is illustrated, according to some embodiments. In some embodiments, the needle hub 112 may include a translucent or optically clear polymer material that is sufficiently rigid to withstand forces of catheterization. As such, a user may visualize interior surfaces, spaces, and components of the needle hub 112 during use. In some embodiments, the needle hub 112 may include the proximal end 114 having the proximal opening 120, the distal end 116 having a distal opening 122, and an interior 118 interposed between the proximal end 114 and the distal end 116.

In some embodiments, the interior 118 may include a distal inner diameter 119, which may be approximately equal to an outer diameter of needle 70 such that a fluid tight interface or seal is provided between needle 170 and the distal inner diameter 119. In some embodiments, the interior 118 may include a proximal inner diameter 121 that is greater than the outer diameter of needle 70 such that a fluid (i.e., blood) may gather and flow between the outer surface of needle 70 and the proximal inner diameter 121. In some embodiments, a portion of the proximal inner diameter 121 may include a flashback chamber 190 of needle hub 112.

In some embodiments, the needle 170 may include a distal opening 177 and a notch 180 in fluid communication with distal opening 177 via a portion of a lumen 178 of the needle 170. In some embodiments, the notch 180 may be formed in and through a sidewall 182 of the needle 170, such that the notch 180 forms a pathway 181 through the sidewall 182. Accordingly, the distal opening 177 may be in fluid communication with the interior 118 via the notch 180.

In some embodiments, the flashback chamber 190 may include a portion of the proximal inner diameter 121 in fluid communication with the notch 180. In these and other embodiments, blood entering the lumen 178 via the distal opening 177 may flow through the lumen 178, exit the needle 170 via notch 180, and flow into flashback chamber 190. In some embodiments, a small spacing or fluid tight seal between the outer surface of the needle 170 and the distal inner diameter 119 prevents blood from flowing therebetween.

In some embodiments, the needle assembly 100 may include a plug 150, which may be fixedly seated within proximal opening 120. In some embodiments, the plug 150 may include an aperture 156 for receiving the proximal end 174 of needle 170. In some embodiments, the proximal opening 176 of the lumen 178 may be accessible at the proximal end 154, and the small spacing or fluid tight seal is provided between aperture 156 and the outer surface of needle 170 to prevent blood within flashback chamber 190 from flowing between aperture 156 and the outer surface of needle 170. In some embodiments, the plug 150 may include an outer surface, which may include a first portion 158 in contact with a proximal inner surface 123 of interior 118. In some embodiments, the plug 150 may include a second portion 159 defining or forming at least one surface of flashback chamber 190. In some embodiments, the second portion 159 may define or form a proximal end surface of the flashback chamber 190. In some embodiments, the second portion 159 may define or form an inner diameter of the flashback chamber 190.

In a closed system, blood flowing through an introducer needle and into a flashback chamber generally produces a positive pressure within the lumen of the needle and within the flashback chamber, thereby reducing or preventing the flow of blood into the flashback chamber. This effect is enhanced when the volume of a lumen of the introducer needle is further reduced by a presence of a guidewire within the lumen. This buildup of positive pressure prevents the user from visualizing advancement of the blood within the flashback chamber, thereby reducing the benefits of using a flashback chamber to confirm proper placement of the distal tip of the catheter during catheterization. Thus, the first portion 158 of the plug 150 may include one or more vents 160, which may be configured to enable airflow through or around the plug 150 to relieve positive pressure within the flashback chamber 190. In some embodiments, the vents 160 may permit air, but not blood, to exit therethrough. As such, the catheter system 10 may provide a facilitate observable flashback, which may facilitate accurate placement of the catheter 15 and reduce a risk of transfixing a vein.

In some embodiments, the first portion 158 of the plug 150 may be modified to include the vents 160. In some embodiments, the vents 160 may include channels interposed between the outer surface of the plug 150 and the proximal inner surface 123 of the interior 118. In some embodiments, the vents 160 may provide a fluid pathway 161 between the plug 150 and the proximal inner surface 123 of the interior 118. In some embodiments, the vents 160 may be configured to prevent or relieve positive pressure within the flashback chamber 190 by providing an access for air to bypass the plug 150 into the exterior environment via a gap 163 provided between a proximal outer surface 157 of the plug 150 and a proximal inner surface 125 of the needle hub 112. In some embodiments, the gap 163 may be annular.

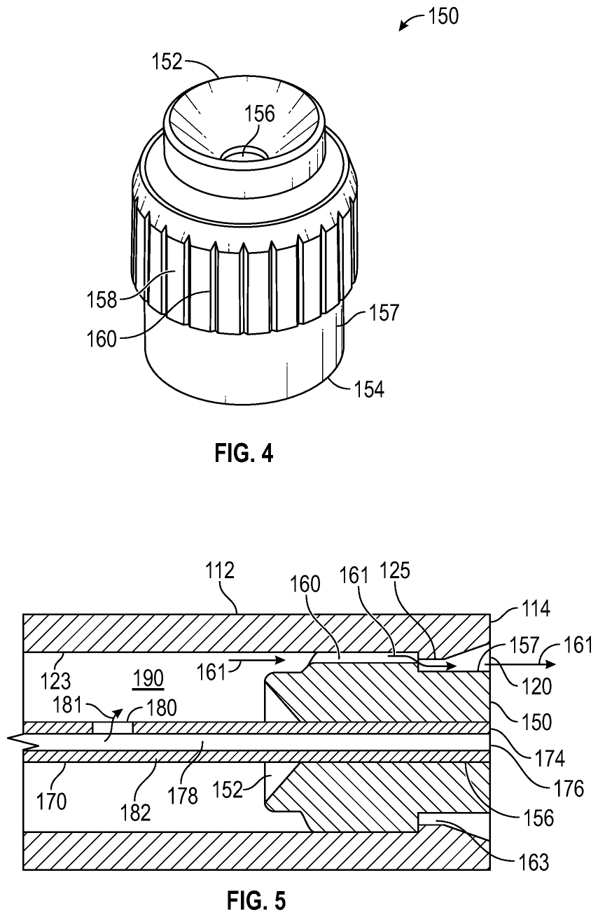

In some embodiments, the gap 163 may be in fluid communication with the fluid pathway 161. In some embodiments, the vents 160 may be constructed by removing material from the first portion 158, which may result in multiple parallel grooves or channels. A close-up perspective view of the plug 150 is provided in FIG. 4, according to some embodiments. In other embodiments, the vents 160 may be formed as channels through the plug 150 rather than on the outer surface of plug 150.

In some embodiments, a rate at which air flows through the fluid pathway 161 of the vents 160 in the plug 150 may be adjusted by manufacturing the plug 150 to include a greater or lesser number of vents 160 or by changing the cross-sectioned area of the vents 160. Thus, in some embodiments the rate at which air and/or fluid flows through the lumen 178 and the flashback chamber 190 may be increased by manufacturing the plug 150 to have either an increased number of the vents 160 or with vents 160 having a greater cross-sectioned area. Conversely, in other embodiments, the rate at which air flows through the lumen 178 and the flashback chamber 190 may be decreased by manufacturing the plug 150 with either a decreased number of the vents 160 or with the vents 160 having a lesser cross-sectioned area.

One having skill in the art will appreciate that the blood pressure of the patient may be largely responsible for the rate at which blood flows through lumen 178 and flashback chamber 190, and therefore also may be largely responsible for the rate at which air within the lumen 178 and the flashback chamber 190 exits through the vents 160. As such, in some embodiments, the flow rate through the catheter system 10 may be affected by a combined effective hydraulic diameter of all flow paths. Thus, in some embodiments, the hydraulic diameter of the vents 160 may be modified to increase or decrease the rate of flow through the lumen 178 and the flashback chamber 190. In other embodiments, the hydraulic diameter of the vents 160 may be decreased thereby resulting in substantially reduced or stopped flow therethrough. The governing equation for controlling the flow rate through the vents 160 is given in Equation 1, where BP is the blood pressure, A is the surface area of vents 160, σ is the surface tension of the blood, and P is the perimeter of the vents 160.

BP(A)=σ(P) Equation 1

Thus, according to Equation 1, when the perimeter of the vents 160 is small, the vents 160 will allow air venting, but will prevent blood flow due to the relatively high surface tension (σ) of blood. Therefore, by adjusting the variables of Equation 1, a desired flow rate may be achieved. Thus, based on the size and/or number of the vents 160 around the plug 150, blood flow may provide customized, controlled, and predictable air flow around the plug 150, thereby providing a desired observable flashback while preventing blood from exiting the proximal opening 120 of the needle hub 112. Accordingly, in some embodiments the vents 160 may be designed to allow the flow of air and stop the flow of blood.

In some embodiments, a number of the vents 160 may be between 1 and 40. In other embodiments, the number of the vents 160 may be between 1 and 20. In some embodiments, the number of the vents 160 may be six or more. In some embodiments, the number of the vents 160 may be five or fewer vents 160. In some embodiments, the vents 160 may have a cross-sectional area between about 0.000007 to 0.00004 inches². In other embodiments, the vents 160 may have a cross-sectional area between about 0.00001 to 0.00003 inches². In other embodiments, the vents 160 may have a cross-sectional area of about 0.00002 inches². For instance, in some embodiments, the vents 160 may have a height of about 0.001 to 0.003 inches and a width of about 0.010 inches. In other embodiments, the vents 160 may have a height of about 0.002 to 0.003 inches and a width of about 0.005 inches.

In some embodiments, the vents 160 may include a cross-sectional area to permit blood flow through the lumen 178 or through the flashback chamber 190 at a rate between about 10 to 200 ml/hr. In some embodiments, the cross-sectional area of the vents 160 can permit blood to flow through the lumen 178 or the flashback chamber 190 at a rate between about 15 to 150 ml/hr. In some embodiments, the cross-sectional area of vents 160 can permit blood to flow through lumen 178 or flashback chamber 190 at a rate between at a rate between about 50 to 100 ml/hr. At these rates, the rate of blood flow into the flashback chamber 190 can be paced to provide a clinician with adequate time to correctly locate the catheter within a patient's blood vessel. Accordingly, in some embodiments, vents 160 have a cross-sectional area greater than 0.00003 inches², a cross-sectional area greater than 0.00004 inches², a cross-sectional area of about 0.0001 inches², or a cross-sectional area of about 0.001 inches².

In some embodiments, the flow rate of blood through lumen 178 is affected by the presence of a guidewire inserted through lumen 178. Accordingly, in some embodiments, a gauge of the guidewire may be selected to provide a desire rate of blood flow through lumen 178. In some embodiments a gauge of the needle 170 may be selected to provide a cross-sectioned area between the inner surface of the needle 170 and the outer surface of the guidewire to provide a desired rate of blood flow through lumen 178. In some embodiments, an inner diameter of a distal portion of the lumen 178 of the needle 170 may be greater than an inner diameter of a proximal portion of the lumen 178 of the needle 170.

In some embodiments, the notch 180 may separate the distal and proximal portions of lumen 178. In some embodiments, the inner diameter of the distal portion of the lumen 178 may permit blood flow between the distal portion of the lumen and the outer surface of the guidewire located within the distal portion of the lumen. In some embodiments, the inner diameter of the proximal portion of the lumen 178 may prevent blood flow between the proximal portion of the lumen 178 and the outer surface of the guidewire located within the proximal portion of the lumen 178.

In some embodiments, the guidewire may include a distal portion, which may include a smaller outer diameter. In some embodiments, the guidewire may include a proximal portion, which may include a larger outer diameter compared to the distal portion of the guidewire. In some embodiments, the notch 180 may be disposed proximate and/or distal to the proximal portion of the guidewire, such that blood is permitted to flow between distal portion of the guidewire and the lumen 178 of the needle 170, and blood is prevented from flowing between the proximal portion of the guidewire and the lumen 178 of the needle 170. In some embodiments, an outer diameter of the guidewire may be increased by placing a portion of the guidewire in a sleeve. In some embodiments, an inner diameter of lumen 178 may be decreased by lining a portion of lumen 178 with a sleeve.

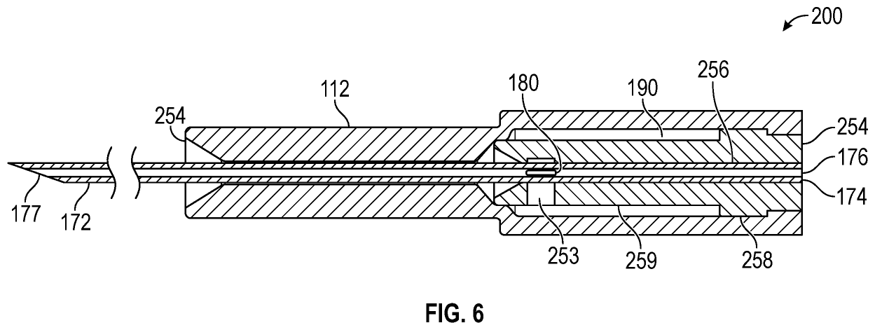

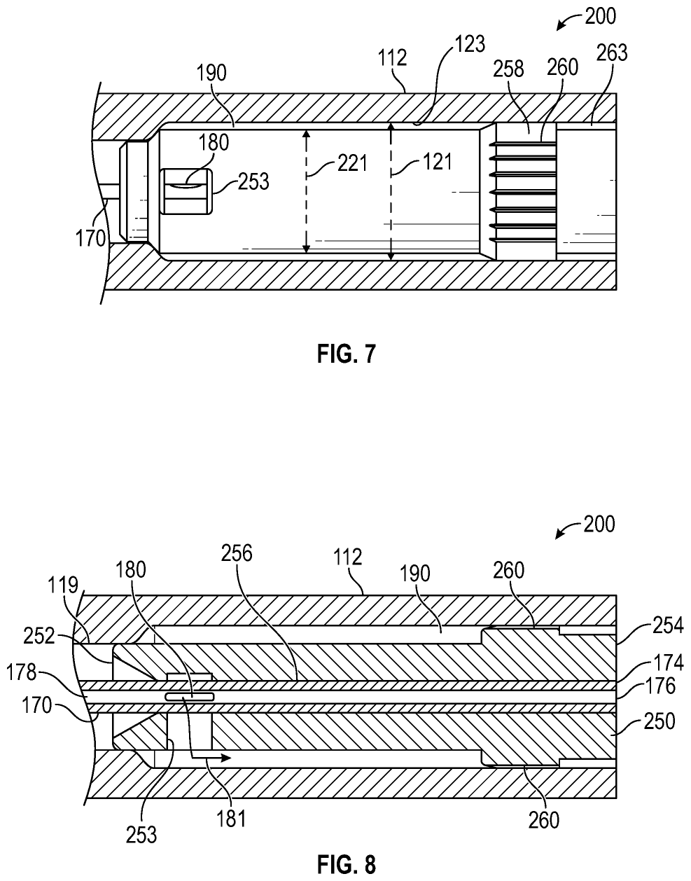

Referring now to FIGS. 6-8, a needle assembly 200 is illustrated, according to some embodiments. In some embodiments, the needle assembly 200 may include a plug 250, which may include a first portion 258 in contact with the proximal inner surface 123 of the interior 118. In some embodiments, the plug 250 may include a second portion 259, which may define at least one surface of the flashback chamber 190. In some embodiments, the first portion 258 may include one or more vents 260, which may be configured to enable airflow through or around the plug 250 to relieve positive pressure within the flashback chamber 190. In some embodiments, the vents 260 may permit air, but not blood, to exit therethrough.

In some embodiments, the needle assembly 200 may be similar or identical to the needle assembly 100 in terms of one or more features and/or operation. In further detail, in some embodiments, the plug 250 may be similar or identical to the plug 150 in terms of one or more features and/or operation. As another example, in some embodiments, the vents 260 may be similar or identical to the vents 160 in terms of one or more features and/or operation.

In some embodiments, the second portion 259 may include an outer diameter 221 that is less than the proximal inner diameter 121 of the interior 118. In some embodiments, the flashback chamber 190 may include the space between the second portion 259 and the proximal inner surface 123 of the interior 118. In some embodiments, placement of the plug 250 within the interior 118 may reduce the volume of the flashback chamber 190, thereby reducing the volume of blood needed to visualize the advancement of the blood within the flashback chamber 190. In some embodiments, the second portion 259 may be elongated to entirely span the proximal inner surface 123 of the interior 118, such that the distal end 252 of the plug 250 forms a fluid tight seal with the distal inner diameter 119 of needle hub 112, thereby forming the distal end of the flashback chamber 190.

In some embodiments, the second portion 259 may include a contrasting color configured to enhance the user's visualization of blood with flashback chamber 190. For example, in some embodiments, the second portion 259 may include an opaque hue that contrasts the red hue of blood. In further detail, the second portion 259 may include a white, yellow, green or blue hue. In some embodiments, the second portion 259 may include two or more hues that contrast with the red hue of blood. In some embodiments, the second portion 259 may include two alternating hues configured to provide a scale by which the user may gauge the speed of the advancement of the blood within the flash chamber 190. In some embodiments, the second portion 259 may include a color pattern by which a user may gauge the speed of the advancement of the blood within chamber 190.

In some embodiments, the plug 250 may include a window 253, which may form a fluid pathway between the aperture 256 of the plug 250 and the flashback chamber 190, such that the window 253 is in fluid communication with the flashback chamber 190. In some embodiments, the window 253 may be positioned in proximity to the distal end 252 of the plug 250 such that the notch 180 of the needle 170 may be positioned within the window 253 when the proximal end 174 of the needle 170 is aligned with the proximal end 254 of the plug 250. Thus, a pathway 181 is provided between the notch 180 and the flashback chamber 190 via the window 253. In some embodiments, a fluid tight seal may be provided between remaining outer surfaces of the needle 170 and the aperture 256 to prevent blood from leaking therebetween. In some embodiments, the plug 250 may include no more than one window 253. In some embodiments, the plug 250 may include two or more windows 253.

In some embodiments, when a guidewire is inserted through the proximal opening 176 of the lumen 178 of the needle 170, blood may be permitted to flow between the inner surface of the lumen 178 and the outer surface of the guidewire along the distal portion of the lumen 178 such that the blood exits the notch 180 and flows through the window 253, however blood may be substantially prevented from flowing through the portions of the lumen 178 and the aperture 256 between the window 253 and the proximal ends 174 and 254, respectively. Accordingly, in some embodiments, the catheter system 10 is configured for use with the guidewire, and flashback confirmation is provided via a vented flashback chamber.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A needle assembly, comprising:
a needle hub configured to couple to a catheter assembly, wherein the needle assembly comprises a proximal end, a distal end, and an interior interposed therebetween, wherein the proximal end of the needle hub comprises a proximal opening, wherein the distal end of the needle hub comprises a distal opening;
a plug, comprising an outer surface, a distal end, a proximal end, and an aperture interconnecting the distal end of the plug and the proximal end of the plug, wherein the outer surface comprises a first portion in contact with an inner surface of the interior, wherein the first portion comprises a vent extending between the distal and proximal ends of the plug and providing a fluid pathway between the outer surface of the plug and the inner surface of the interior, wherein the fluid pathway interconnects the interior and the proximal opening of the needle hub;
an introducer needle, comprising a distal end and a proximal end, wherein the proximal end of the introducer needle is secured to the proximal end of the needle hub via the aperture, and the distal end of the introducer needle extends beyond the distal end of the needle hub, wherein a proximal opening of the introducer needle is accessible at the proximal end of the needle hub such that a guidewire may be threaded through a lumen of the introducer needle via the proximal opening of the introducer needle and extend out through a distal opening of the introducer needle, wherein the introducer needle comprises a notch forming a pathway through a sidewall of the introducer needle and in fluid communication with the lumen; and
a flashback chamber in fluid communication with the notch and defining a space between the inner surface and a second portion of the outer surface.

2. The needle assembly of claim 1, wherein the flashback chamber is positioned between the distal end of the plug and the distal end of the needle hub.

3. The needle assembly of claim 1, wherein the fluid pathway comprises a cross-sectional area that permits the passage of air but not blood.

4. The needle assembly of claim 3, wherein the cross-sectional area is between 0.0001 to 0.0003 inches².

5. The needle assembly of claim 1, wherein the first portion of the outer surface comprises a plurality of vents that includes the vent, each of the plurality of vents providing a fluid pathway between the first portion of the outer surface and the inner surface of the interior.

6. The needle assembly of claim 1, comprising a fluid tight seal between the distal end of the plug and the inner surface of the interior.

7. The needle assembly of claim 6, wherein the distal end of the plug comprises a window in fluid communication with the flashback chamber.

8. The needle assembly of claim 7, wherein the notch is positioned within the window such that the notch is in fluid communication with the flashback chamber via the window.

9. The needle assembly of claim 7, comprising a fluid tight seal between an outer surface of the introducer needle and the aperture.

10. The needle assembly of claim 1, wherein the second portion of the outer surface comprises an outer diameter that is less than an inner diameter of the interior.

11. The needle assembly of claim 1, wherein the notch is positioned within the flashback chamber.

12. The needle assembly of claim 1, wherein the distal end of the plug is inwardly chamfered.

13. The needle assembly of claim 1, further comprising a fluid tight seal between an outer surface of the introducer needle and the aperture.

14. The needle assembly of claim 1, further comprising a gap between the proximal end of the plug and the inner surface of the interior in proximity to the proximal opening of the needle hub.

15. The needle assembly of claim 14, wherein the gap is interposed between the vent and the proximal opening of the needle hub.

16. A guidewire assisted catheter system, comprising:
a catheter assembly, comprising a catheter adapter comprising a catheter extending distally from a distal end of the catheter adapter; and
a needle assembly according to claim 1.

17. The guidewire assisted catheter system of claim 16, wherein fluid pathway comprises a cross-sectional area that permits the passage of air but not blood.

18. The guidewire assisted catheter system of claim 17, wherein the cross-sectional area is between 0.0001 and 0.0003 inches².

19. The guidewire assisted catheter system of claim 16, comprising a fluid tight seal between the distal end of the plug and the inner surface of the interior.

20. The guidewire assisted catheter system of claim 19, wherein the distal end of the plug comprises a window in fluid communication with the flashback chamber.
